(19) United States
(12) Patent Application Publication
(10) Pub. No.: US 2023/0335274 A1
(43) Pub. Date: Oct. 19, 2023

(54) A REMOTE MEDICAL EXAMINATION SYSTEM AND METHOD

(71) Applicant: Tyto Care LTD., Netanya (IL)

(72) Inventors: Eyal BYCHKOV, Hod Hasharon (IL); Marina VASILEVSKY, Modi'in (IL); Yariv FISHMAN, Ra'anana (IL); Pavel VASILEV, Petah Tikva (IL); Elad LACHMANOVICH, Modi'in (IL)

(21) Appl. No.: 17/791,219

(22) PCT Filed: Jan. 5, 2021
(86) PCT No.: PCT/IL2021/050011

§ 371 Date: Jul. 7, 2022

Related U.S. Application Data
(60) Provisional application No. 62/958,329, filed on Jan. 8, 2020.

(51) Int. Cl. G16H 40/67; G16H 80/00

(52) U.S. Cl. CPC G16H 40/67; G16H 80/00

(57) ABSTRACT

A system for performing one or more remote medical examinations of a patient, the system comprising a display and at least one processing resource configured to perform the following for at least one remote medical examination of the remote medical examinations: receive, during the remote medical examination, from a remote medical data acquisition device, first data generated from medical data acquired by a sensor from a body of the patient, the first data having a first quality measure; receive second data generated from the medical data, the second data having a second quality measure, higher than the first quality measure; display the first data on the display during the remote medical examination, to a medical practitioner, thereby enabling the medical practitioner to provide the navigation or placement instructions: and upon receiving a high-quality data request from the medical practitioner, display the second data on the display, to the medical practitioner.

### TECHNICAL FIELD

The invention relates to a remote medical examination system and method.

### BACKGROUND

Telemedicine solutions enable medical practitioners to provide remote diagnosis and/or treatment of patients via telecommunication technologies. For this purpose, the medical practitioners rely on medical data provided by the patient herself (by voice and/or text) and on medical data acquired by medical data acquisition device/s that are designed to collect medical data from the patient’s body. The medical data acquisition devices comprise sensors that collect the medical data.

One of the challenges in telemedicine is navigating the medical data acquisition device to a required location with respect to the patient’s body and placing the medical data acquisition device correctly with respect to the patient’s body. One solution to this challenge includes sending data acquired by the sensors of the medical data acquisition device to a remote medical practitioner workstation (remote from the medical data acquisition device) for display to a remote medical practitioner, thereby enabling the medical practitioner to provide the user of the medical data acquisition device with instructions for navigating and/or placing the medical data acquisition device in a desirable manner for acquiring medical data that can be used for diagnosis. However, the bandwidth available for sending data from the medical data acquisition device to the remote medical practitioner workstation is expensive and, in some cases, also limited, especially when it is required to be sent in real-time. Therefore, the medical data can be sent in a first quality for navigation and placement purposes, and in a second quality, higher than the first quality, for diagnosis purposes. However, in such cases, the medical practitioner may mistakenly attempt to provide a diagnosis based on the medical data of the first quality, which may result in an inaccurate diagnosis, or, in worst cases, even in an erroneous diagnosis.

There is thus a need in the art for a new remote medical examination system and method.

### GENERAL DESCRIPTION

In accordance with a first aspect of the presently disclosed subject matter, there is provided a system for performing one or more remote medical examinations of a patient, the system comprising a display and at least one processing resource configured to perform the following for at least one remote medical examination of the remote medical examinations: receive, during the remote medical examination, from a remote medical data acquisition device, first data generated from medical data acquired by a sensor of the remote medical data acquisition device from a body of the patient, the first data having a first quality measure usable for providing navigation or placement instructions for navigating the medical data acquisition device to a desired spatial disposition with respect to the patient’s body or for changing a placement of the medical data acquisition device; receive second data generated from the medical data, the second data having a second quality measure, higher than the first quality measure, usable for diagnosing a medical condition of the patient; display the first data on the display during the remote medical examination, to a medical practitioner using the system, thereby enabling the medical practitioner to provide the navigation or placement instructions; and upon receiving a high-quality data request from the medical practitioner, display the second data on the display, to the medical practitioner, thereby enabling the medical practitioner to diagnose the medical condition of the patient.

In some cases, the at least one processing resource is further configured to perform the following for at least one remote medical examination of the remote medical examinations: perform a measure upon the medical practitioner attempting to diagnose the medical condition of the patient before at least part of the second data being provided to the medical practitioner by the processing resource.

In some cases, the measure is one or more of the following: (a) providing an alert; (b) preventing the medical practitioner from providing a diagnosis of the medical condition of the patient; (c) updating a log associated with a diagnosis provided by the medical practitioner to indicated that the diagnosis is not based on the second data; (d) associated the diagnosis with an indication that the diagnosis is not based on the second data.

In some cases, the second data is received upon the medical practitioner pressing a link requesting downloading the second data from a remote storage location.

In some cases, the medical data includes one or more of the following: images, sound streams, or video recordings.

In some cases, the first data is generated by performing a lossy compression of the medical data.

In some cases, the second data is one of: (a) the medical data unmanipulated, (b) a first result of a pre-processing of the medical data or (c) a second result of a lossless compression of the medical data.

In some cases, the second data is received by the system after the first data is received by the system.

In some cases, the at least one processing resource is further configured to perform the following for at least one remote medical examination of the remote medical examinations: store the second data on a storage device associated with the system for future use by the medical practitioner.

In some cases, the at least one processing resource is further configured to perform the following for at least one remote medical examination of the remote medical examinations: monitor a time lapsing from acquisition of the medical data: and provide an alert to a user of the system upon the monitored time failing to meet a criterion based on a maximal allowed time until review, by the medical practitioner, of the medical data.

In some cases, the criterion is (a) lapsing of the maximal allowed time, or (b) lapsing of a pre-determined part of the maximal allowed time.

In some cases, the pre-determined part of the maximal allowed time is determined as (a) a given percentage of the maximal allowed time, or (b) a subtraction of a given amount of time from the maximal allowed time.

In accordance with a second aspect of the presently disclosed subject matter, there is provided a method for performing one or more remote medical examinations of a patient, the method comprises performing the following for at least one remote medical examination of the remote medical examinations: receiving, by a processing resource, during the remote medical examination, from a remote medical data acquisition device, first data generated from medical data acquired by a sensor of the remote medical data acquisition device from a body of the patient, the first data having a first quality measure usable for providing navigation or placement instructions for navigating the medical data acquisition device to a desired spatial disposition with respect to the patient’s body or for changing a placement of the medical data acquisition device; receiving, by the processing resource, second data generated from the medical data, the second data having a second quality measure, higher than the first quality measure, usable for diagnosing a medical condition of the patient, displaying the first data on a display during the remote medical examination, to a medical practitioner using the system, thereby enabling the medical practitioner to provide the navigation or placement instructions; and upon receiving a high-quality data request from the medical practitioner, displaying the second data on the display, to the medical practitioner, thereby enabling the medical practitioner to diagnose the medical condition of the patient.

In some cases, the method further comprises performing the following for at least one remote medical examination of the remote medical examinations: performing a measure upon the medical practitioner attempting to diagnose the medical condition of the patient before at least part of the second data being provided to the medical practitioner by the processing resource.

In some cases, the measure is one or more of the following: (a) providing an alert; (b) preventing the medical practitioner from providing a diagnosis of the medical condition of the patient; (c) updating a log associated with a diagnosis provided by the medical practitioner to indicated that the diagnosis is not based on the second data; (d) associated the diagnosis with an indication that the diagnosis is not based on the second data.

In some cases, the second data is received upon the medical practitioner pressing a link requesting downloading the second data from a remote storage location.

In some cases, the medical data includes one or more of the following: images, sound streams, or video recordings.

In some cases, the first data is generated by performing a lossy compression of the medical data.

In some cases, the second data is one of: (a) the medical data unmanipulated, (b) a first result of a pre-processing of the medical data or (c) a second result of a lossless compression of the medical data.

In some cases, the second data is received by the system after the first data is received by the system.

In some cases, the method further comprises performing the following for at least one remote medical examination of the remote medical examinations: storing the second data on a storage device for future use by the medical practitioner.

In some cases, the method further comprises performing the following for at least one remote medical examination of the remote medical examinations: monitoring a time lapsing from acquisition of the medical data; and providing an alert to a user of the system upon the monitored time failing to meet a criterion based on a maximal allowed time until review, by the medical practitioner, of the medical data.

In some cases, the criterion is (a) lapsing of the maximal allowed time, or (b) lapsing of a pre-determined part of the maximal allowed time.

In some cases, the pre-determined part of the maximal allowed time is determined as (a) a given percentage of the maximal allowed time, or (b) a subtraction of a given amount of time from the maximal allowed time.

In accordance with a third aspect of the presently disclosed subject matter, there is provided a non-transitory computer readable storage medium having computer readable program code embodied therewith, the computer readable program code, executable by a processing resource to perform a method for performing one or more remote medical examinations of a patient, the method comprises performing the following for at least one remote medical examination of the remote medical examinations: receiving, by a processing resource, during the remote medical examination, from a remote medical data acquisition device, first data generated from medical data acquired by a sensor of the remote medical data acquisition device from a body of the patient, the first data having a first quality measure usable for providing navigation or placement instructions for navigating the medical data acquisition device to a desired spatial disposition with respect to the patient’s body or for changing a placement of the medical data acquisition device; receiving, by the processing resource, second data generated from the medical data, the second data having a second quality measure, higher than the first quality measure, usable for diagnosing a medical condition of the patient; displaying the first data on a display during the remote medical examination, to a medical practitioner using the system, thereby enabling the medical practitioner to provide the navigation or placement instructions; and upon receiving a high-quality data request from the medical practitioner, displaying the second data on the display, to the medical practitioner, thereby enabling the medical practitioner to diagnose the medical condition of the patient

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1 is a block diagram schematically illustrating one example of a system for performing a medical examination of a patient by a remote medical practitioner, in accordance with the presently disclosed subject matter,

FIG. 2 is a block diagram schematically illustrating one example of a medical practitioner workstation, in accordance with the presently disclosed subject matter,

FIG. 3 is a flowchart illustrating one example of a sequence of operations carried out for performing a remote examination, in accordance with the presently disclosed subject matter, and

FIG. 4 is a flowchart illustrating one example of a sequence of operations carried out for monitoring a service level, in accordance with the presently disclosed subject matter.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “receiving”, “displaying”, “performing”, “storing”, “providing”, “preventing” or the like, include action and/or processes of a computer that manipulate and/or transform data into other data, said data represented as physical quantities, e.g. such as electronic quantities, and/or said data representing the physical objects. The terms “computer”, “processor”, “processing resource” and “controller should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field programmable gate array (FPGA), an application specific integrated circuit (ASIC), etc.), a group of multiple physical machines sharing performance of various tasks, virtual servers coresiding on a single physical machine, any other electronic computing device, and/or any combination thereof.

The operations in accordance with the teachings herein may be performed by a computer specially constructed for the desired purposes or by a general-purpose computer specially configured for the desired purpose by a computer program stored in a non-transitory computer readable storage medium. The term “non-transitory” is used herein to exclude transitory, propagating signals, but to otherwise include any volatile or non-volatile computer memory technology suitable to the application.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

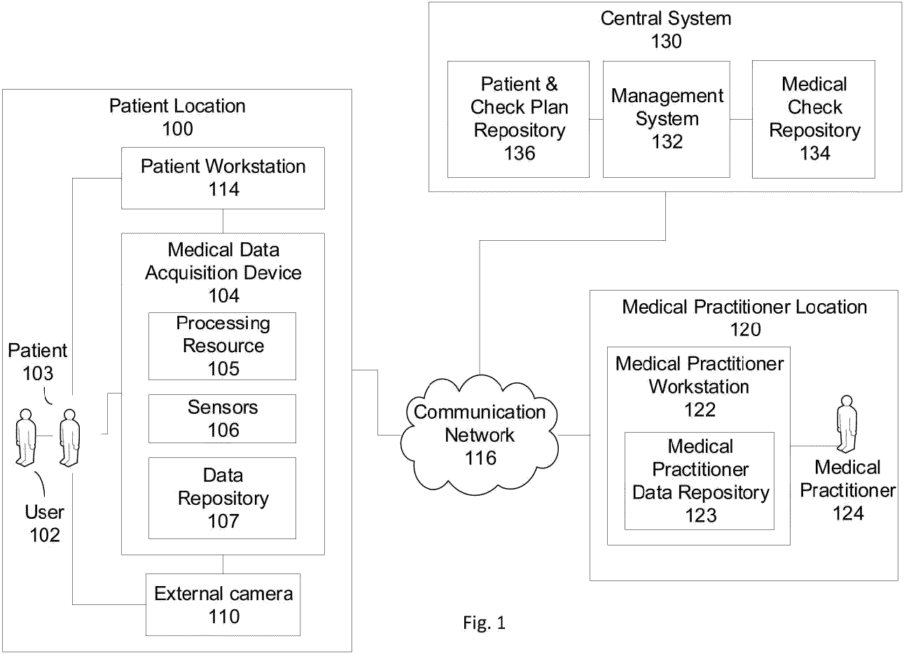

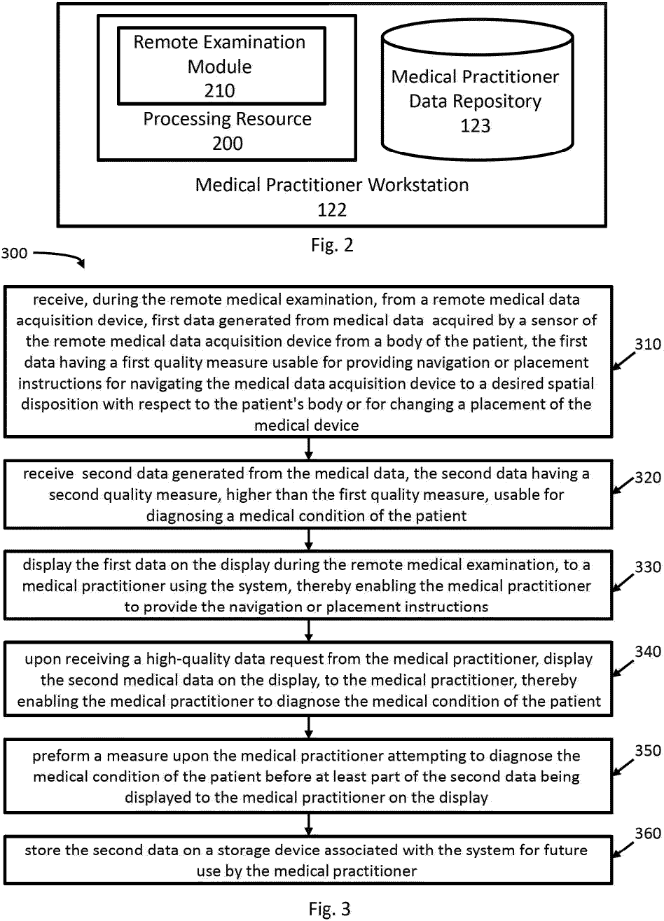

In embodiments of the presently disclosed subject matter, fewer, more and/or different stages than those shown in FIG. 3 may be executed. In embodiments of the presently disclosed subject matter one or more stages illustrated in FIG. 3 may be executed in a different order and/or one or more groups of stages may be executed simultaneously. FIGS. 1 and 2 illustrate a general schematic of the system architecture in accordance with an embodiment of the presently disclosed subject matter. Each module in FIGS. 1 and 2 can be made up of any combination of software, hardware and/or firmware that performs the functions as defined and explained herein. The modules in FIGS. 1 and 2 may be centralized in one location or dispersed over more than one location. In other embodiments of the presently disclosed subject matter, the system may comprise fewer, more, and/or different modules than those shown in FIGS. 1 and 2.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

Bearing this in mind, attention is drawn to FIG. 1, a block diagram schematically illustrating one example of a system for performing a medical examination of a patient by a remote medical practitioner, in accordance with the presently disclosed subject matter. A user 102 and a patient 103 (a person or an animal whose medical examination is required) are located at patient location 100, and a medical practitioner 124 is located at a medical practitioner location 120, remote from the patient location 100. In fact, according to the presently disclosed subject matter, the medical practitioner 124 is located at the medical practitioner location 120 that is remote from the patient location 100 so that the medical practitioner 124 does not have direct access to the patient 103 (e.g. it is not located in the same room with the patient 103, nor in any other form of vicinity thereto, so that the medical practitioner 124 cannot himself hold the medical data acquisition device 104 and place it on the body of the patient 103 for acquiring medical data therefrom). In some cases, the medical practitioner 124 can be located in a different room/floor/building/street/city/state/country/continent than the patient 103.

In light of the fact that the medical practitioner 124 is located at a different location than the patient 103, the user 102 is required to operate the medical data acquisition device 104 for acquiring medical data from the patient’s 103 body. In this respect, it is to be noted that the user 102 can be the patient 103 whose medical examination is required (in such cases, even though user 102 and patient 103 are shown as separate entities in the drawings, they are in fact the same entity). In other cases, the user 102 can be another person (other than patient 103) that will operate the medical data acquisition device 104 for acquiring medical data from the patient’s 103 body, as further detailed herein. In some cases, the user 102 is not a medical practitioner, i.e. the user 102 is not a person specifically trained to acquire medical data from the patient’s 103 body, nor is be qualified to diagnose a medical condition of the patient 103 based on medical data acquired from the patient’s body.

Attention is drawn to the components within the patient location 100:

The medical data acquisition device 104 comprises (or is otherwise associated with) at least one processing resource 105. Processing resource 105 can be one or more processing units (e.g. central processing units), microprocessors, microcontrollers (e.g. microcontroller units (MCUs)) or any other computing/processing device, which are adapted to independently or cooperatively process data for controlling relevant medical data acquisition device 104 resources and for enabling operations related to medical data acquisition device 104 resources.

Medical data acquisition device 104 further comprises one or more sensors 106 (e.g. camera/s, microphone/s, a thermometer, depth camera/s, an otoscope, a blood pressure sensor, an electrocardiogram (ECG), an ultrasound sensor, an acoustic sensor, a blood saturation sensor, etc.), including at least one sensor capable of acquiring medical data from the patient’s 103 body, based on which the medical practitioner 124 can diagnose a medical condition of the patient 103. The medical data can be, for example, body temperature, blood pressure, blood saturation, ECG measurements, audio signals (e.g. of the heart operations or of the lungs), ultrasound signals (e.g. of the heart, of the intestines, etc.), acoustic measurements, body tissue electrical resistance, hardness of body tissues, a heartrate, an image or a video recording of a body organ or a portion of a body organ (whether internal body organ or external body organ), a 3D representation of one or more body organs or portions thereof (whether internal body organ or external body organ), a blood sample analysis, urine samples, throat cultures, saliva samples, or any other parameter associated with one or more physiological characteristic of a patient, based on which diagnosis can be provided.

In some cases, medical data acquisition device 104 can further comprise, or be otherwise associated with, a data repository 107 (e.g. a database, a storage system, a memory including Read Only Memory - ROM, Random Access Memory - RAM, or any other type of memory, etc.) configured to store data, including inter alia patient-related data relating to one or more patients 103 and various medical data acquired from such patients 103 body (e.g. data acquired during a medical examination of the patients using the medical data acquisition device 104), various configuration parameters of the sensor(s) 106, check plans for patient 103 (e.g. defining medical examinations to be performed on patient 103), threshold parameters (e.g. defining required quality levels for various types of measurements), etc. In some cases, data repository 107 can be further configured to enable retrieval and/or update and/or deletion of the stored data. It is to be noted that in some cases, data repository 107 can be distributed across multiple locations, whether within the medical data acquisition device 104 and/or within patient location 100 and/or within central system 130 and/or within medical practitioner location 120 and/or elsewhere. It is to be noted, that in some cases, the relevant information relating to the patient 103 can be loaded into data repository 107 before performing medical examination of the 103 (e.g. upon beginning of a medical examination and/or periodically and/or upon an entity such as the medical practitioner 124 requesting the information).

It is to be noted that in some cases, the medical data acquisition device 104 can be a handheld device, and at least the processing resource 105 and the sensors 106 can be comprised within a housing of the medical data acquisition device 104, that can optionally be a handheld device. In some cases, the sensors can be comprised within removably attachable units configured to be attached to the medical data acquisition device 104. In some cases, the sensors can be external to the medical data acquisition device 104 and in such cases, it may communicate with the medical data acquisition device 104 via a wired connection and/or via a wireless connection (e.g. a WiFi connection).

It is to be further noted that in some cases, medical data acquisition device 104 can further comprise one or more speakers for providing audio recordings to the user 102 (e.g. recordings of a medical practitioner 124 instructing the user 102 how to perform medical examinations, voice instructions generated by the medical data acquisition device 104 instructing the user 102 how to perform medical examinations, etc.). Medical data acquisition device 104 can further comprise a microphone for recording sounds, including voices (e.g. of the user 102 and/or patient 103), in the vicinity of the medical data acquisition device 104, e.g. during medical examinations conducted using the medical data acquisition device 104. Medical data acquisition device 104 can further comprise a display for providing visual output to the user 102 (e.g. a video recording of a remote medical practitioner 124, computer generated instructions instructing the user 102 how to perform medical examinations, indications of quality of an acquired measurement, etc.).

In some cases, medical data acquisition device 104 can communicate, directly, or indirectly, with patient workstation 144 and/or with medical practitioner workstation 122 and/or with central system 130, through communication network 116 (e.g. the Internet), via wired or wireless communication. It is to be noted that such communication can alternatively or additionally be performed utilizing other known communication alternatives, such as a cellular network, Virtual Private Network (VPN), Local Area Network (LAN), etc.

In some cases, a camera 110 can also be located at the patient location 100. Camera 110 (also referred to as “external camera 110”) is external to medical data acquisition device 104, in the sense that it is not comprised within the housing of the medical data acquisition device 104. Camera 110 is preferably movable irrespectively of medical data acquisition device 104. Camera 110 is operable to capture visible light, and to generate images or video based on light it captures. Camera 110 may additionally, or alternatively, be sensitive to other parts of the electromagnetic spectrum near the visible spectrum (e.g. to infrared radiation, such as near IR radiation). Camera 110 may be sensitive to the entire visible spectrum (e.g. a commercial-off-the-shelf camera, such as a DSLR camera, a smartphone camera, a webcam camera), or only to a part of it. In some cases, the camera 110 can be a depth camera, capable of generating a 3D representation of the examination process.

Camera 110 is oriented toward the examined patient’s 103 body location, in at least some of the time during which medical data acquisition device 104 acquires medical data from the patient’s 103 body. Especially, camera 110, when oriented toward the examined patient’s 103 body location (as described), is operable to acquire one or more images (that can optionally form a video) which includes at least a part of the patient’s 103 body and at least part of the medical data acquisition device 104 when medical data acquisition device 104 (or one or more of the sensors 106) is adjacent to the examined patient 103 body location. Accordingly, images capture by the camera 110 include at least part of the medical data acquisition device 104 and a location on the body of the patient 103 which is currently examined thereby.

In some cases, a patient workstation 114 can also be located at the patient location 100. Patient workstation 114 can be any computer, including a personal computer, a portable computer, a smartphone or any other apparatus with appropriate processing capabilities, including an apparatus which can be, for example, specifically configured for that purpose. The patient workstation 114 can be operated by user 102, for receiving inputs therefrom (e.g. questions to answers, various identification information, etc.), and/or for providing output thereto (showing operational instructions for operating the medical data acquisition device 104, etc.). In some cases, patient workstation 114 can communicate with medical data acquisition device 104 and/or with medical practitioner workstation 122 and/or with central system 130, through communication network 116 (e.g. the Internet), via wired or wireless communication. It is to be noted that such communication can alternatively or additionally be performed utilizing other known communication alternatives, such as a cellular network, Virtual Private Network (VPN), Local Area Network (LAN), etc. It is to be noted that in some cases, patient workstation 114 can comprise the camera 110, and in a more specific example, patient workstation 114 can be a smartphone and camera 110 can be a camera of the smartphone. It is to be noted that in some cases, the processing resources of the patient workstation 114, or of any other computer (located at the patient location 100 or elsewhere), can perform some of the tasks described with reference to processing resource 105 of the medical data acquisition device 104.

Attention is drawn to the components within the medical practitioner location 120:

A medical practitioner workstation 122 is located at the medical practitioner location 120. Medical practitioner workstation 122 can be any computer, including a personal computer, a portable computer, a smartphone or any other apparatus with appropriate processing capabilities, including an apparatus which can be, for example, specifically configured for that purpose. The medical practitioner workstation 122 can receive inputs from the medical practitioner 124 (e.g. instructions and/or questions to be provided to the user 102 and/or patient 103, etc.), and/or provide output to the medical practitioner 124 (showing the medical data acquired by the medical data acquisition device 104, etc.). In some cases, medical practitioner workstation 122 can communicate with medical data acquisition device 104 and/or patient workstation 114 and/or central system 130, through communication network 116 (e.g. the Internet), via wired or wireless communication. It is to be noted that such communication can alternatively or additionally be performed utilizing other known communication alternatives, such as a cellular network, VPN, LAN, etc. In some cases, medical practitioner workstation 122 can communicate with one or more other medical practitioner workstations 122, e.g. when a first medical practitioner operating the medical practitioner workstation 122 is interested in obtaining a second opinion, optionally relating to a certain diagnosis provided by the first medical practitioner from another medical practitioner.

In some cases, medical practitioner workstation 122 can further comprise, or be otherwise associated with, a medical practitioner data repository 123 (e.g. a database, a storage system, a memory including Read Only Memory - ROM, Random Access Memory - RAM, or any other type of memory, etc.) configured to store data, including inter alia medical data acquired by the medical data acquisition device 104 (optionally including also various metadata relating to such medical data), and other patient-related data relating to one or more patients 103. In some cases, medical practitioner data repository 123 can be further configured to enable retrieval and/or update and/or deletion of the stored data. It is to be noted that in some cases, medical practitioner data repository 123 can be distributed across multiple locations, whether within the medical practitioner location 120 and/or within central system 130 and/or elsewhere. It is to be noted, that in some cases, the relevant information relating to a given examined patient 103 can be loaded into data repository 123 before performing medical examination of a patient 103 (e.g. upon beginning of a medical examination and/or periodically and/or upon an entity such as the medical practitioner 124 requesting the information). In some cases, the medical data can include Electronic Health Records (EHR) data relating to one or more patients 103. In some cases, the EHR data can be obtained through an interface (e.g. over the communication network 116) to a remote EHR system.

In some cases, medical practitioner workstation 122 can communicate with patient workstation 144 and/or with medical data acquisition device 104 and/or with central system 130, through communication network 116 (e.g. the Internet), via wired or wireless communication. It is to be noted that such communication can alternatively or additionally be performed utilizing other known communication alternatives, such as a cellular network, Virtual Private Network (VPN), Local Area Network (LAN), etc.

In some cases, a central system 130 can exist, for allowing a distributed approach in which medical data and/or other patient-related data can be received by the central system 130 from multiple patient locations 100 and transferred by it to multiple medical practitioner locations 120. Thus, in case the transmitted medical data and/or other patient-related data is received at central system 130, it can be stored in medical check repository 134 and management system 132 can transmit it to a specific medical practitioner location 120 (e.g. via communication network 116 such as the Internet). In some cases, management system 132 can also manage other processes such as, subscribing patients, planning scheduling of patients to available medical practitioners, etc.

It is to be noted that central system 130 is optional to the solution and that central system 130 can be part of the medical practitioner workstation 122. In addition, the communication between the patient workstation 114 and/or the medical data acquisition device 104, and the medical practitioner workstation 122 can be performed directly without the use of or need for a central system 130.

In those cases where a central system 130 exists, it can comprise patient & check plan repository 136 in which various patient-related data, relating to one or more patients 103, is maintained. Such patient-related data can include, for example, patient identification number, patient name, patient age, patient contact details, patient medical record data (such as the patients EHR, information of patient’s diseases, sensitivities to medicines, etc.), check plans data (as further detailed below), etc. Central system 130 can further comprise a medical examination repository 134 in which one or more of the following can be stored: (a) medical data acquired by medical data acquisition device 104 (optionally including also various metadata relating to such medical data), (b) user-provided data, provided by the user 102, e.g. using the patient workstation 114, including type-ins and/or voice recording and/or additional info provided by user 102 and relating to the patient 103, and (c) diagnosis data provided by a medical practitioner diagnosing the patient 103. The medical data and/or the user-provided data, can include, for example, voice recordings and/or video recordings and/or values of one or more of the following parameters: body temperature, blood pressure, blood saturation, electrocardiogram (ECG) measurements, audio signals (e.g. of the heart operations or of the lungs), ultrasound signals (e.g. of the heart, of the intestines, etc.), acoustic measurements, body tissue electrical resistance, hardness of body tissues, a heartrate, an image or a video recording of a body organ or a portion of a body organ (whether internal body organ or external body organ), a blood sample analysis, a 3D representation of one or more body organs or portions thereof (whether internal body organ or external body organ), urine samples, throat cultures, saliva samples, or any other parameter associated with one or more physiological characteristic of a patient, based on which diagnosis can be provided. In some cases, one or more of the parameter values can be associated with metadata, such as a timestamp indicative of the time in which the parameter value was acquired, location data indicative of the location at which the parameter value was acquired (e.g. geographical coordinates, WiFi Internet Protocol (IP) address, etc.), a sensor type, information enabling identification of a specific sensor with which the parameter value was acquired. Inertial Navigation System (INS) and/or pressure sensor and/or room humidity and/or room temperature and/or patient orientation and/or room ambient noise level readings acquired during acquisition of the parameter value.

Central system 130 can further comprise management system 132 configured to forward medical data acquired by the medical data acquisition device 104 (whether in a raw form, or any processed version of the raw data acquired by the medical data acquisition device 104) and relating to a patient 103, and optionally other patient-related data relating to the patient 103, to a selected medical practitioner workstation 122 (for example an available medical practitioner workstation 122 or medical practitioner workstation 122 with the shortest queue, e.g. in case where no medical practitioner, out of a plurality of medical practitioners, is currently available). It is to be noted that when providing a central system 130, there may be more than one medical practitioner location 120 and more than one medical practitioner 124 as central system 130 can allow the distributed approach in which data (e.g. medical data and/or other patient-related) can be received by the central system 130 from multiple patient locations 100 and transferred by it to multiple medical practitioner locations 120.

Having described the various components in the patient location 100, in the medical practitioner location 120 and the central system 130, attention is drawn to two exemplary modes of operation of the medical data acquisition device 104: an on-line mode and an off-line mode.

In an on-line mode, a medical examination of the patient 103 is conducted while the medical practitioner 124 is actively involved in the process. In such operation mode, the medical practitioner 124 can be provided with a video or a sequence of images, based on which the medical practitioner 124 provides the user 102 with instructions for positioning the medical data acquisition device 104 with respect to the patient’s 103 body. In addition, the medical practitioner 124 can provide the user 102 with instructions for performing a current medical examination (other than positioning instructions) and/or with instructions for performing other medical examinations as part of the medical examination flow. In some cases, the instructions can be audible instructions, acquired by a microphone on the medical practitioner location (e.g. a microphone connected to the medical practitioner workstation 122), and provided to the user 102 via a speaker in the patient location 100 (e.g. a speaker of the medical data acquisition device 104, a speaker of the patient workstation 114, or any other speaker which provides sounds that the user 102 can hear). Additionally, or alternatively to the audible instructions, the instructions can be video instructions provided via a display in the patient location 100 (e.g. a display of the medical data acquisition device 104, a display of the patient workstation 114, or any other display visible to the user 102).

The video that is provided to the medical practitioner 124 can be acquired by a camera comprised within the medical data acquisition device 104 (e.g. one of the sensors 106 can be a camera used for this purpose), and in such case, the medical practitioner 124 can view the part of the patient’s body to which the camera is aimed. In additional, or alternative cases, the video can be acquired by an external camera 110 external to the medical data acquisition device 104, and in such cases, the medical practitioner 124 can view the patient 103 and the medical data acquisition device 104 in the same frame. In any case, based on the camera’s view, the medical practitioner 124 can provide the user 102 with maneuvering instructions for navigating the medical data acquisition device 104 to a desired spatial disposition with respect to the patient’s 103 body. In some cases, the video can be accompanied by a sound recording acquired using a microphone located at the patient location 100 (e.g. a microphone of the medical data acquisition device 104, a microphone of the patient workstation 114, or any other microphone that can acquire a sound recording of sounds at the patient location 100)

Upon arrival of the medical data acquisition device 104 to the desired spatial disposition (from which the medical data can be acquired) with respect to the patient’s 103 body, the medical practitioner 124 can instruct the user 102 to acquire the medical data, or it can operate the sensors 106 himself to acquire the medical data. In some cases, the medical practitioner 124 can also remotely control various parameters of the sensors 106, e.g. through medical practitioner workstation 122.

It is noted that medical data acquisition device 104 can be located outside the body of the patient when acquiring the medical data. Nevertheless, in some cases some parts of medical data acquisition device 104 may enter the body of the patient (e.g. a needle penetrating the skin and/or a blood vessel, a sensor entering a body orifice such as the ear or the mouth, and so on). Even in such cases, the greater part of medical data acquisition device 104 can be located outside the body at the time of measurement.

The medical data acquired by the medical data acquisition device 104 can be transmitted to the medical practitioner workstation 122 (directly, or through the patient workstation 114 and/or through the central system 130 where it can be stored in the medical check repository 134 in association with the patient 103 from which the medical data was acquired), where it can be stored in medical practitioner data repository 123 in association with the patient 103 from which the medical data was acquired.

The medical practitioner 124 (e.g. a doctor, a nurse, a medic, etc., including any other person with the know-how and skill to acquire and/or analyze medical data), located at medical practitioner location 120, can review the acquired medical data, for example using medical practitioner workstation 122. It is to be noted that patient workstation 114, medical practitioner workstation 122 and central system 130 can include a display (e.g. LCD screen), and a keyboard or any other suitable input/output devices.

In some cases, medical practitioner 124 can provide feedback data (e.g. by transmitting corresponding instructions to patient workstation 114 and/or to medical data acquisition device 104) to user 102, such as a diagnosis, one or more prescriptions, or instructions to perform one or more additional medical examinations. Alternatively, or additionally, medical practitioner 124 can transmit feedback data to central system 130, which, in turn, can optionally transmit the feedback data to patient workstation 114 and/or to the medical data acquisition device 104 (e.g. via the communication network 116).

In some cases, the medical data acquisition device 104 and/or the patient workstation 114 can be configured to provide the user 102 with an indication of a quality of a signal acquired by the sensors. In such cases, the medical data acquisition device 104 and/or the patient workstation 114 can be configured to determine the signal quality and display an appropriate indication on a display visible by the user 102 (e.g. a display of the medical data acquisition device 104 and/or a display of the patient workstation 114). In some cases, upon the signal quality not meeting pre-defined thresholds, the medical data acquisition device 104 and/or the patient workstation 114 can be configured to provide the user 102 with instructions for improving the acquired signal quality (e.g. instructions to reposition the medical data acquisition device 104, instructions to seduce ambient noise, etc.).

In an off-line mode, a medical examination of the patient 103 is conducted while no medical practitioner 124 is actively involved in the process. In such operation mode, the medical data acquisition device 104 can provide the user 102 with audio and/or video navigation instructions for navigating the medical data acquisition device 104 to s desired spatial disposition with respect to the patient’s 103 body. The navigation instructions can be determined by the medical data acquisition device 104 and/or by the patient workstation 114 using information obtained from an Inertial Navigation System (INS), that can optionally be part of the sensors 106, and/or using matching of reference points within reference images and images acquired by a camera comprised within the medical data acquisition device 104 and/or by external camera 110. The navigation instructions can be provided via a speaker and/or a display of the medical data acquisition device 104 and/or of the patient workstation 114 and/or of any other device located near the user 102 in a manner that enables a user to hear and/or see the navigation instructions.

Upon arrival of the medical data acquisition device 104 to the desired spatial disposition (from which the medical data can be acquired) with respect to the patient’s 103 body, the user 102 can operate the medical data acquisition device 104 to acquire medical data, or alternatively, the medical data acquisition device 104 can acquire the medical data automatically.

In some cases, the medical data acquisition device 104 and/or the patient workstation 114 can be configured to provide the user 102 with an indication of a quality of a signal acquired by the sensors. In such cases, the medical data acquisition device 104 and/or the patient workstation 114 can be configured to determine the signal quality and display an appropriate indication on a display visible by the user 102 (e.g. a display of the medical data acquisition device 104 and/or a display of the patient workstation 114). In some cases, upon the signal quality not meeting pre-defined thresholds, the medical data acquisition device 104 and/or the patient workstation 114 can be configured to provide the user 102 with instructions for improving the acquired signal quality (e.g. instructions to reposition the medical data acquisition device 104, instructions to reduce ambient noise, etc.).

It is noted that medical data acquisition device 104 can be located outside the body of the patient when acquiring the medical data. Nevertheless, in some cases some parts of medical data acquisition device 104 may enter the body of the patient (e.g. a needle penetrating the skin and/or a blood vessel, a sensor entering a body orifice such as the ear or the mouth, and so on). Even in such cases, the greater part of medical data acquisition device 104 can be located outside the body at the time of measurement.

The medical data acquired by the medical data acquisition device 104 can be transmitted to a medical practitioner workstation 122 (directly, or through the patient workstation 114 and/or through the central system 130 where it can be stored in the medical check repository 134 in association with the patient 130 from which the medical data was acquired), where it can be stored in medical practitioner data repository 123 in association with the patient 130 from which the medical data was acquired.

The medical practitioner 124 (e.g. a doctor, a medic, etc., including any other entity (human or computerized) with the know-how and skill to acquire and/or analyze medical data), located at medical practitioner location 120, can review the acquired medical data, for example using a display and/or a speaker and/or any other suitable output device of the medical practitioner workstation 122. It is to be noted that patient workstation 114, medical practitioner workstation 122 and central system 130 can include a display (e.g. LCD screen), and a keyboard or any other suitable input/output devices.

In some cases, medical practitioner 124 can provide feedback data (e.g. by transmitting corresponding instructions to patient workstation 114 and/or to medical data acquisition device 104) to user 102, such as a diagnosis, one or more prescriptions, or instructions to perform one or more additional medical examinations. Alternatively, or additionally, medical practitioner 124 can transmit feedback data to central system 130, which, in turn, can optionally transmit the feedback data to patient workstation 114 (e.g. via the communication network 116). As indicated herein, the feedback data can be provided to the user 102 via an output device (e.g. a display, a speaker, etc.) of the medical data acquisition device 104 and/or of the patient workstation, or of any other device that is capable of providing the respective output to the user 102.

It is to be noted that in some cases, the medical practitioner data repository 123 and/or the data repository 107, and or the medical check repository 134 and/or the patient & check plan repository 136 can be the same single data repository, whether distributed or not, that is accessible by all relevant entities.

Turning to FIG. 2, there is shown a block diagram schematically illustrating one example of a medical practitioner workstation, in accordance with the presently disclosed subject matter.

As detailed with reference to FIG. 1, medical practitioner workstation 122 comprises, or is otherwise associated with, a medical practitioner data repository 123. The medical practitioner data repository 123 can store, and enable retrieval or deletion of, medical data acquired by the sensors 106 of the medical data acquisition device 104. In some cases, the medical data that is acquired by the sensors can be received in two (or more) different channels, while the medical data sent via each channel has a different quality.

For example, a first channel can include the medical data in a first quality, that is sufficient for enabling the medical practitioner 124 to provide the user 102 with navigation and/or positioning instructions for navigating the medical data acquisition device 104 to a desired spatial disposition with respect to the patient’s 103 body, and/or for placing the medical data acquisition device 104 at a desired placement (e.g. a desired pressure level) with respect to the patient’s 103 body. A second channel can include the medical data in a second quality, better/higher than the first quality, that is more likely to enable the medical practitioner 124 to accurately diagnose the patient 103 (e.g. enabling the medical practitioner 124 to determine if noises in readings which include a recording of the lungs are related to a medical condition of the patient 103 or simply reading noise, or to determine if a suspicious area on the patient’s 103 skin is dirt or a mole that requires analysis). For example, the first data can be compressed in a lossy manner in which some information is lost, while the second data can be compressed in a lossless manner in which no information is lost (as the originally acquired medical data can be reconstructed therefrom) or in a less lossy manner so that less information is lost when compared to the first data. As another example, the first data can be of a first resolution, degraded with respect to the originally acquired medical data, while the second data is of a second resolution, higher than the first resolution. It is to be noted that the second data consumes more bandwidth upon its transmittal than the first data. It is to be noted that compression ratio and resolution are two exemplary parameters that are associated with the quality of the data, but other parameters exist that also have an effect on the quality of the data.

When reference is made to quality of the medical data, it is to be noted that when the medical data is images/video, the quality refers to image quality (resolution, frame rate, and/or other parameters that are associated with the quality of the images/videos), whereas when the medical data is a sound recording, the quality refers to the sound recording quality (accuracy, fidelity, intelligibility, sampling rate, and/or any other parameters that are associated with the quality of the sound recording).

The medical practitioner workstation 122 further comprises at least one processing resource 200. Processing resource 200 can be one or more processing units (e.g. central processing units), microprocessors, microcontrollers or any other computing devices or modules, including multiple and/or parallel and/or distributed processing units, which are adapted to independently or cooperatively process data for controlling relevant resources of the medical practitioner workstation 122 and for enabling operations related to resources of the medical practitioner workstation 122.

The processing resource 200 comprises a remote examination module 210 configured to perform a remote examination process, as further detailed herein with respect to FIG. 3, attention to which is now drawn.

FIG. 3 is a flowchart illustrating one example of a sequence of operations carried out for performing a remote examination, in accordance with the presently disclosed subject matter.

According to certain examples of the presently disclosed subject matter, medical practitioner workstation 122 (also referred to herein as “system”) can be configured to perform a remote examination process 300, e.g. utilizing the remote examination module 210.

For this purpose, medical practitioner workstation 122 can be configured to receive, during a remote medical examination of a patient 103, from a remote medical data acquisition device 104 (directly or indirectly, e.g. via proxy servers, etc.), first data generated from medical data acquired by a sensor 106 of the remote medical data acquisition device 104 from a body of the patient 103 (block 310). The medical data can be images acquired by a sensor that includes a camera, sound streams acquired by a sensor that includes microphone, or video recordings acquired by a sensor that includes a camera.

The first data, that is generated from the medical data acquired by the sensor 106 of the medical data acquisition device 104, has a first quality measure and it is usable for providing a user 102, operating the medical data acquisition device 104, with navigation and/or placement instructions for navigating the medical data acquisition device to a desired spatial disposition (e.g. in six degrees of freedom) with respect to the patient’s body, or for changing a placement of the medical data acquisition device (e.g. instructing the user to apply more pressure when pressing a sensor of the medical data acquisition device 104, used to acquire the medical data, onto the body of the patient 103). In some cases, the first data is generated by performing a lossy compression of the medical data, which results in the first data having a degraded quality in comparison to the quality of the originally acquired medical data acquired by the sensor 106.

The fact that the first data is smaller in size (thereby requiring less network bandwidth upon transmittal) than the raw data acquired by the sensor 106 (and also smaller in size than the second data), enables communication of the first data to the medical practitioner workstation 122 faster than communicating the raw data or the second data to the medical practitioner workstation 122. This enables the medical practitioner 124 to provide the user 102 with closer to real-time instructions based on the first data that arrives to the medical practitioner 124 than if the medical practitioner had to wait to the second data or the raw data to arrive to the medical practitioner workstation 122.

In addition to receiving the first data at block 310, medical practitioner workstation 122 is further configured to receive second data generated from the medical data (the same medical data from which the first data received at block 310 is generated), the second data having a second quality measure, higher than the first quality measure of the first data (block 320). It is to be noted, in this respect, that using the second data for diagnosis purposes results in increasing the likelihood of providing an accurate diagnosis of a medical condition of the patient 103 as compared to using the first data for this purpose. However, as indicated herein, the first data can have a lower quality measure than the second data which will suffice for providing the user 102 with navigation/placement instructions for navigating/placing the medical data acquisition device 104 to/at a desired spatial disposition with respect to the patient’s 103 body.

In some cases, the second data is received by the medical practitioner workstation 122 automatically, without requiring any action of the medical practitioner 124. in other cases, the second data is received by the medical practitioner workstation 122 upon the medical practitioner 124 requesting receipt thereof (e.g. by pressing a link provided thereto and accessible via the medical practitioner workstation 122, e.g. via a user interface thereof, for downloading the second data) from a remote storage location thereof (e.g. from data repository 107 or from the medical check repository 134, or from any other location in which it is stored).

In some cases, the second data is the medical data as acquired by the sensor 106, without any manipulation (the raw data acquired thereby). In other cases, the second data is a result of a pre-processing of the medical data, which includes, for example, noise filtering, marking of points of interest, or any other type of pre-processing. Additionally, or alternatively, the second data can be a result of a lossless compression of the medical data, which does not result in degradation of the quality of the second data in comparison to the raw medical data as acquired by the sensor (as it can be fully reconstructed from the lossless compression result).

It is to be noted that the second data is received by the medical practitioner workstation 122 after the first data is received by the medical practitioner workstation 122.

Medical practitioner workstation 122 is further configured to display the first data on a display thereof (in case the first data is image/video data) or output the data via a speaker (in case the first data is sound data), during the remote medical examination of the patient 103, to a medical practitioner 124 using the medical practitioner workstation 122, thereby enabling the medical practitioner 124 to provide the navigation or placement instructions to the user 102, optionally with an overlay or any other type of marking indicating that the displayed data is not intended to be used for diagnosis purposes (block 330). The first data can be displayed/outputted to the medical practitioner 124 in real-time, or near real-time, so that the instructions provided to the user 102 are relevant and usable during operation of the medical data acquisition device 104 in an on-line mode (e.g. as detailed with reference to FIG. 1).

upon receiving a high-quality data request from the medical practitioner 124, medical practitioner workstation 122 is configured to display/output the second data on the display or through the speaker, to the medical practitioner 124, thereby enabling the medical practitioner 124 to diagnose the medical condition of the patient 103 (block 340). As indicated herein, the second data is of a higher quality that the first data that is displayed/outputted to the medical practitioner 124 at block 330 for navigation/placement purposes.

In some cases, the medical practitioner 124 may attempt to diagnose a medical condition of the patient 103 based on the first data, without reviewing the second data. As indicated herein, the first data is of lower quality when compared to the second data. Accordingly, diagnosing a medical condition of the patient 103 based thereon is disadvantageous, and in some cases even dangerous as it increases the likelihood of erroneous diagnosis. Therefore, the medical practitioner workstation 122 is further configured to perform a measure upon the medical practitioner 122 attempting to diagnose the medical condition of the patient 103 before at least part of the second data is provided to the medical practitioner 124 by the medical practitioner workstation 122 (block 350).

The measure can be one or more of the following:
(a) providing an alert to the medical practitioner 124 and/or to the user 102 indicating that the diagnosis is not based on the second data but on the first data that is of lower quality:
(b) preventing the medical practitioner 124 from providing a diagnosis of the medical condition of the patient 103 until the second data, or at least part thereof, is provided to the medical practitioner 124;
(c) updating a log associated with a diagnosis provided by the medical practitioner 124 to indicated that the diagnosis is not based on the second data; or
(d) associated the diagnosis with an indication that the diagnosis is not based on the second data.

In some cases, the medical practitioner workstation 122 is further configured to store the second data (and optionally also the first data) on a storage device associated therewith (e.g. medical practitioner data repository 123, medical check repository 134, or any other data repository accessible by the medical practitioner workstation 122) for future use by the medical practitioner 122, or by any other medical practitioner that desires to obtain the second data (or the first data if available) (block 360).

It is to be noted that, with reference to FIG. 3, some of the blocks can be integrated into a consolidated block or can be broken down to a few blocks and/or other blocks may be added. Furthermore, in some cases, the blocks can be performed in a different order than described herein (for example, block 360 can be performed before block 350, block 350 can be performed before block 340, block 330 can be performed before block 320, etc.). It is to be further noted that some of the blocks are optional. It should be also noted that whilst the flow diagram is described also with reference to the system elements that realizes them, this is by no means binding, and the blocks can be performed by elements other than those described herein.

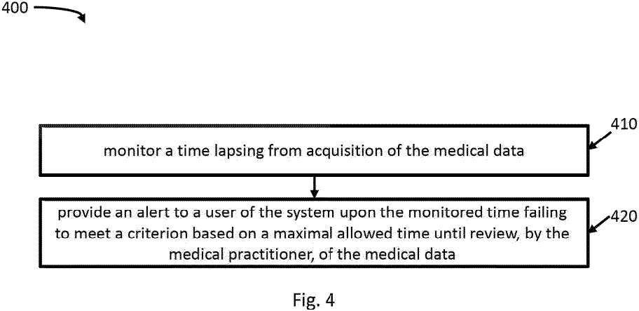

Turning to FIG. 4, there is shown a flowchart illustrating one example of a sequence of operations carried out for monitoring a service level, in accordance with the presently disclosed subject matter.

According to certain examples of the presently disclosed subject matter, medical practitioner workstation 122 and/or central system 130 and/or medical data acquisition device and/or any other computerized device can be configured to perform a service level monitoring process 400, e.g. utilizing a suitable module of a respective processing resource/s thereof (e.g. the remote examination module 210 of processing resource 200).

For this purpose, any system that executes the service level monitoring process 300, referred to herein as service level monitoring system, can be configured to monitor a time lapsing from acquisition of the medical data (block 410). This can be achieved by activating a timer, or by periodically comparing the acquisition time of the medical data and the current time.

The service level monitoring system can provide an alert to a user thereof (e.g. user 102, medical practitioner 124, or any other user) upon the monitored time failing to meet a criterion based on a maximal allowed time until review, by the medical practitioner, of the medical data (block 420). The maximal allowed time can be defined by a Service Level Agreement (SLA). In some cases, the maximal allowed time can be determined using rules that are based on severity of a medical situation of the patient. The severity can be determined, for example, by automatically analyzing the medical data acquired from the patient 103, or by an indication of a severity level provided directly by the user 102 based on her own assessment.

The criterion can be (a) lapsing of the maximal allowed time, or (b) lapsing of a pre-determined part of the maximal allowed time, while the pre-determined part of the maximal allowed time can be determined as (a) a given percentage of the maximal allowed time, or (b) a subtraction of a given amount of time from the maximal allowed time.

Accordingly, medical data can be acquired from a patient 103 at a certain point in time. The acquired medical data (or versions thereof, such as the first data or second data detailed inter alia with reference to FIG. 3) can be sent to medical service provider such as an Health Maintenance Organization (HMO) which is responsible to assign a medical practitioner 124 to attend to diagnosing the medical data. The medical practitioner 124 can be assigned to diagnose a plurality of patients 103 (optionally by a plurality of HMOs), while each patient 103 has its own SLA defining a maximal allowed time until review, by the medical practitioner 124, of medical data acquired therefrom. There is a risk that the medical practitioner 124 will fail to meet the SLA requirements, and therefore having the ability to monitor the time lapsing from acquisition of the medical data is beneficial.

It is to be noted that, with reference to FIG. 4, the blocks can be integrated into a consolidated block or can be broken down to a few blocks and/or other blocks may be added. It should be also noted that whilst the flow diagram is described also with reference to the system elements that realizes them, this is by no means binding, and the blocks can be performed by elements other than those described herein.

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed method. The presently disclosed subject matter further contemplates a machine-readable memory tangibly embodying a program of instructions executable by the machine for executing the disclosed method.

1. A system for performing one or more remote medical examinations of a patient, the system comprising a display and at least one processing resource configured to perform the following for at least one remote medical examination of the remote medical examinations:
receive, during the remote medical examination, from a remote medical data acquisition device, first data generated from medical data acquired by a sensor of the remote medical data acquisition device from a body of the patient, the first data having a first quality measure usable for providing navigation or placement instructions for navigating the medical data acquisition device to a desired spatial disposition with respect to the patient’s body or for changing a placement of the medical data acquisition device;
receive second data generated from the medical data, the second data having a second quality measure, higher than the first quality measure, usable for diagnosing a medical condition of the patient;
display the first data on the display during the remote medical examination, to a medical practitioner using the system, thereby enabling the medical practitioner to provide the navigation or placement instructions; and
upon receiving a high-quality data request from the medical practitioner, display the second data on the display, to the medical practitioner, thereby enabling the medical practitioner to diagnose the medical condition of the patient.

2. The system of claim 1, wherein the at least one processing resource is further configured to perform the following for at least one remote medical examination of the remote medical examinations:
perform a measure upon the medical practitioner attempting to diagnose the medical condition of the patient before at least part of the second data being provided to the medical practitioner by the processing resource.

3. The system of claim 2, wherein the measure is one or more of the following: (a) providing an alert; (b) preventing the medical practitioner from providing a diagnosis of the medical condition of the patient; (c) updating a log associated with a diagnosis provided by the medical practitioner to indicated that the diagnosis is not based on the second data; (d) associated the diagnosis with an indication that the diagnosis is not based on the second data.

4. The system of claim 1, wherein the second data is received upon the medical practitioner pressing a link requesting downloading the second data from a remote storage location.

5. The system of claim 1, wherein the medical data includes one or more of the following: images, sound streams, or video recordings.

6. The system of claim 1, wherein the first data is generated by performing a lossy compression of the medical data.

7. The system of claim 1, wherein the second data is one of: (a) the medical data unmanipulated, (b) a first result of a pre-processing of the medical data or (c) a second result of a lossless compression of the medical data.

8. The system of claim 1, wherein the second data is received by the system after the first data is received by the system.

9. (canceled)

10. The system of claim 1, wherein the at least one processing resource is further configured to perform the following for at least one remote medical examination of the remote medical examinations:
monitor a time lapsing from acquisition of the medical data; and
provide an alert to a user of the system upon the monitored time failing to meet a criterion based on a maximal allowed time until review, by the medical practitioner, of the medical data.

11. (canceled)

12. (canceled)

13. A method for performing one or more remote medical examinations of a patient, the method comprises performing the following for at least one remote medical examination of the remote medical examinations:
receiving, by a processing resource, during the remote medical examination, from a remote medical data acquisition device, first data generated from medical data acquired by a sensor of the remote medical data acquisition device from a body of the patient, the first data having a first quality measure usable for providing navigation or placement instructions for navigating the medical data acquisition device to a desired spatial disposition with respect to the patient’s body or for changing a placement of the medical data acquisition device;
receiving, by the processing resource, second data generated from the medical data, the second data having a second quality measure, higher than the first quality measure, usable for diagnosing a medical condition of the patient;
displaying the first data on a display during the remote medical examination, to a medical practitioner using the system, thereby enabling the medical practitioner to provide the navigation or placement instructions; and
upon receiving a high-quality data request from the medical practitioner, displaying the second data on the display, to the medical practitioner, thereby enabling the medical practitioner to diagnose the medical condition of the patient.

14. The method of claim 13, further comprising performing the following for at least one remote medical examination of the remote medical examinations:
performing a measure upon the medical practitioner attempting to diagnose the medical condition of the patient before at least part of the second data being provided to the medical practitioner by the processing resource.

15. The method of claim 14, wherein the measure is one or more of the following: (a) providing an alert; (b) preventing the medical practitioner from providing a diagnosis of the medical condition of the patient; (c) updating a log associated with a diagnosis provided by the medical practitioner to indicated that the diagnosis is not based on the second data; (d) associated the diagnosis with an indication that the diagnosis is not based on the second data.

16. The method of claim 13, wherein the second data is received upon the medical practitioner pressing a link requesting downloading the second data from a remote storage location.

17. The method of claim 13, wherein the medical data includes one or more of the following: images, sound streams, or video recordings.

18. The method of claim 13, wherein the first data is generated by performing a lossy compression of the medical data.

19. The method of claim 13, wherein the second data is one of: (a) the medical data unmanipulated, (b) a first result of a pre-processing of the medical data or (c) a second result of a lossless compression of the medical data.

20. The method of claim 13, wherein the second data is received by the system after the first data is received by the system.

21. (canceled)

22. The method of claim 13, wherein the at least one processing resource is further configured to perform the following for at least one remote medical examination of the remote medical examinations:
monitor a time lapsing from acquisition of the medical data; and
provide an alert to a user of the system upon the monitored time failing to meet a criterion based on a maximal allowed time until review, by the medical practitioner, of the medical data.

23. (canceled)

24. (canceled)

25. A non-transitory computer readable storage medium having computer readable program code embodied therewith, the computer readable program code, executable by a processing resource to perform a method for performing one or more remote medical examinations of a patient, the method comprises performing the following for at least one remote medical examination of the remote medical examinations:
receiving, by a processing resource, during the remote medical examination, from a remote medical data acquisition device, first data generated from medical data acquired by a sensor of the remote medical data acquisition device from a body of the patient, the first data having a first quality measure usable for providing navigation or placement instructions for navigating the medical data acquisition device to a desired spatial disposition with respect to the patient’s body or for changing a placement of the medical data acquisition device;
receiving, by the processing resource, second data generated from the medical data, the second data having a second quality measure, higher than the first quality measure, usable for diagnosing a medical condition of the patient;
displaying the first data on a display during the remote medical examination, to a medical practitioner using the system, thereby enabling the medical practitioner to provide the navigation or placement instructions; and
upon receiving a high-quality data request from the medical practitioner, displaying the second data on the display, to the medical practitioner, thereby enabling the medical practitioner to diagnose the medical condition of the patient.
